(19) United States
(12) Patent Application Publication
(10) Pub. No.: US 2019/0008790 A1
(43) Pub. Date: Jan. 10, 2019

(54) A METHOD FOR THE PREPARATION OF A DELIVERY DRUG DELIVERY SYSTEM AND A COMPOSITION THEREFOR

(71) Applicant: Element Six (UK) Limited, Oxfordshire (GB)

(72) Inventor: Dong WANG, Oxfordshire (GB)

(21) Appl. No.: 16/065,230

(22) PCT Filed: Dec. 19, 2016
(86) PCT No.: PCT/EP2016/081681

§ 371 Date: Jun. 22, 2018

(30) Foreign Application Priority Data
Dec. 30, 2015 (GB) 1523081.6

(51) Int. Cl. A61K 9/51; A61K 9/19; A61K 31/192; A61K 31/085; A61K 31/366; A61K 31/496

(52) U.S. Cl. CPC A61K 9/5192; A61K 9/5115; A61K 9/19; A61K 31/496; A61K 31/085; A61K 31/366; A61K 31/192

(57) ABSTRACT

A method for the preparation of a delivery drug delivery system, the drug having one or more active pharmaceutical ingredients having solubility in water of less than 1 g in 30 ml of water and nano-diamond, comprises the steps of dissolving the active pharmaceutical ingredient(s) into a polar non-aqueous solvent to form a first mixture, dissolving a surfactant in deionized water to form a surfactant solution, adding a plurality of nano-diamond particles to the surfactant solution to disperse the nano-diamond particles in the surfactant solution thereby forming a nano-diamond dispersion, adding the first mixture to the nano-diamond dispersion whilst agitating the dispersion to form a second mixture; and drying the second mixture to produce a dry powder for use as a drug delivery system.

### FIELD

This disclosure relates to a method for the preparation of a delivery drug delivery system and a composition therefor, particularly but not exclusively for use in the delivery of active pharmaceutical ingredients (“APIs”), in particular APIs having low or no solubility in water.

### BACKGROUND

Nano-diamond has been extensively used as a means of delivering active pharmaceutical ingredients. Nano-diamond is non-toxic and biocompatible so is suitable for use in-vivo.

The conventional approach to using nano-diamond as a means for delivering active pharmaceutical ingredients is either to chemically bond (e.g. by means of a carboxylic acid or amine group), or to non-covalently bond (e.g. by van der Waals forces) the active pharmaceutical ingredient on to the surface of the nano-diamond particle. Nano-diamond particles can acquire high levels of charge so therefore have high stability as a colloid.

Ever increasing drives for improved drug delivery mechanisms place ever increasing demands on the materials and methods used. There is therefore a need for a method for the preparation of a delivery drug delivery system and a composition therefor which may be suitable for delivering drugs into the body, in particular drugs that do not have a high solubility in water or are insoluble in water.

### SUMMARY

Viewed form a first aspect there is provided a method for the preparation of a delivery drug delivery system comprising one or more active pharmaceutical ingredients having solubility in water of less than 1 g in 30 ml of water and nano-diamond, the method comprising the steps of:

(a) dissolving the active pharmaceutical ingredient(s) into a polar non-aqueous solvent to form a first mixture;
(b) dissolving a surfactant in deionized water to form a surfactant solution;
(c) adding a plurality of nano-diamond particles to the surfactant solution to disperse the nano-diamond particles in the surfactant solution thereby forming a nano-diamond dispersion;
(d) adding the first mixture to the nano-diamond dispersion whilst agitating the dispersion to form a second mixture; and
(e) drying the second mixture to produce a dry powder for use as a drug delivery system.

Viewed from a second aspect there is provided a composition for medical treatment comprising:

a plurality of nano-diamond particles; and
an active pharmaceutical ingredient having a solubility in water of less than 1 g in 30 ml of water wherein
the composition is a dry powder having an average particle size in the range 500 nm or less when dispersed in deionised water.

### BRIEF DESCRIPTION OF THE DRAWINGS

Versions will now be described by way of example and with reference to the accompanying drawings in which:

FIG. 1 is a flow diagram showing the steps of according to a first example of the method;

FIG. 2 is a plot of the results of an IC₅₀ test for Triclosan™ nanodiamond formulation having an IC₅₀ value of ˜4.5 ppm showing percentage growth compared to positive control against Triclosan™ ppm;

FIG. 3 is a plot of the results of an IC₅₀ test for Triclosan™ in a 50:50 by volume water/ethanol solution having an IC₅₀ value of ˜56 ppm showing percentage growth compared to positive control against Triclosan™ ppm; and

FIGS. 4(a)-(c) show the results of the activity tests described in Example 8 on the powders produced in Example 7 and Comparative Example 7 showing percentage regrowth compared to positive control against mg/ml of Ciprofloxacin.

### DETAILED DESCRIPTION

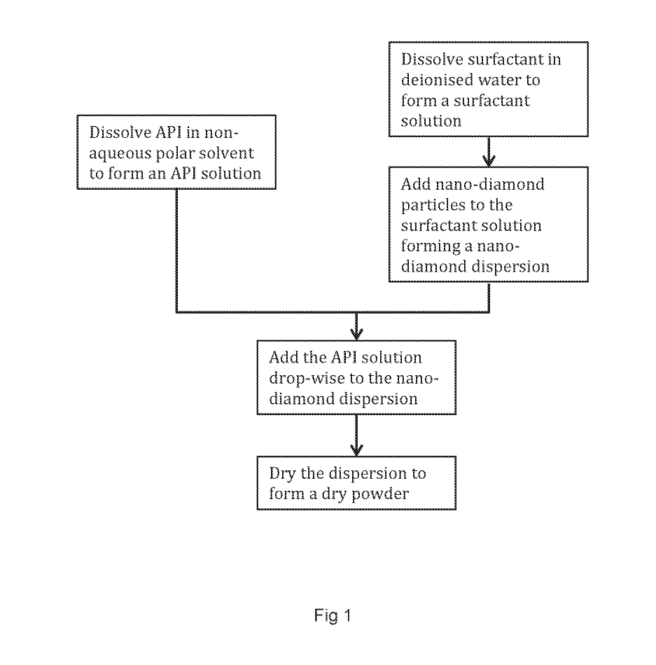

FIG. 1 is a flow diagram of the method steps of a first example for the preparation of a delivery drug delivery system comprising one or more active pharmaceutical ingredients having solubility in water of less than 1 g in 30 ml of water, and nano-diamond. As shown in FIG. 1, the method comprises the steps of dissolving the active pharmaceutical ingredient(s) into a polar non-aqueous solvent to form a first mixture, dissolving a surfactant in deionized water to form a surfactant solution, adding a plurality of nano-diamond particles to the surfactant solution to disperse the nano-diamond particles in the surfactant solution thereby forming a nano-diamond dispersion, adding the first mixture to the nano-diamond dispersion whilst agitating the dispersion to form a second mixture, and drying the second mixture to produce a dry powder for use as a drug delivery system.

In some examples, the first mixture is added drop-wise to the nano-diamond dispersion.

The means of agitation of the nano-diamond dispersion whilst the first mixture is added to the nano-diamond dispersion may be, for example, mechanical means or ultrasonic means.

In some examples, the step of drying is freeze-drying. In other examples, the step of drying is spray-drying or spray granulation.

The resultant dry powder may be re-dispersed in water or other suitable dispersant or mixture of dispersants to facilitate the active pharmaceutical ingredient being delivered as a drug by suitable means.

In some examples, the resultant dry powder is further processed into one or more tablets, capsules, gel or patches for delivery by suitable means.

“Suitable means” includes, but is not limited to, delivery by injection, oral delivery, topical delivery, and ocular delivery.

In some examples, the polar non-aqueous solvent is dimethyl sulfoxide ((CH₃)₂SO), also known as “DMSO”. Other suitable non-aqueous solvents may be used. Examples of other suitable solvents may include, without limitation, 98% ethanol, ethanol, acetonitrile, chlorobenzene, chloroform, cyclohexane, 1,2-dichloroethene, 1,2-dimethoxyethane, N,N-dimethylacetamide, N,N-dimethylformamide, 1,4-dioxane, 2-ethoxyethanol, ethylene glycol, formamide, hexane, methanol, 2-methoxyethanol, methylbutylketone, methylcyclohexane, methylene chloride, N-methylpyrrolidone, nitromethane, pyridine, sulfolane, tetrahydrofuran, tetralin, toluene, trichloroethylene, xylene, acetic acid, heptane, acetone, isobutyl acetate, anisole, isopropyl acetate, 1-butanol, methyl acetate, 2-butanol, 3-methyl-1-butanol, butyl acetate, methylethylketone, tert-butylmethyl ether, methylisobutylketone, cumene, 2-methyl-l-propanol, pentane, 1-pentanol, ethyl acetate, 1-propanol, ethyl ether, 2-propanol, ethyl formate, propyl acetate, and formic acid.

In some examples, the concentration of the active pharmaceutical ingredient in the polar non-aqueous solvent is in the range 1 g/dm³ to 200 g/dm³. In some examples, the lower limit of the concentration range of the active pharmaceutical ingredient in the polar non-aqueous solvent may be 1 g/dm³, alternatively 2 g/dm³, alternatively 5 g/dm³, alternatively 10 g/dm³. In some examples, the upper limit of the concentration range of the active pharmaceutical ingredient in the polar non-aqueous solvent may be 200 g/dm³, alternatively 100 g/dm³, alternatively 75 g/dm³, alternatively 50 g/dm³. In one example, the surfactant is cetyl trimethyl ammonium bromide ((C₁₆H₃₃)N(CH₃)₃Br, also known as hexadecyl-trimethyl-ammonium bromide). Other suitable pharmaceutically acceptable surfactants may also be used.

Other pharmaceutically acceptable surfactants may be used. The surfactant may be non-ionic, anionic, cationic, amphoteric or zwitterionic.

Examples of suitable non-ionic surfactants may include ethoxylated triglycerides; fatty alcohol ethoxylates; alkylphenol ethoxylates; fatty acid ethoxylates; fatty amide ethoxylates; fatty amine ethoxylates; sorbitan alkanoates; ethylated sorbitan alkanoates; alkyl ethoxylates; block copolymers of ethylene oxide and propylene oxide, i.e. poloxamers (available under the trade name Pluronics™); alkyl polyglucosides; stearol ethoxylates; alkyl polyglycosides.

Examples of suitable anionic surfactants may include alkylether sulfates; alkylether carboxylates; alkylbenzene sulfonates; alkylether phosphates; dialkyl sulfosuccinates; sarcosinates; alkyl sulfonates; soaps; alkyl sulfates; alkyl carboxylates; alkyl phosphates; paraffin sulfonates; secondary n-alkane sulfonates; alpha-olefin sulfonates; isethionate sulfonates.

Examples of suitable cationic surfactants may include fatty amine salts; fatty diamine salts; quaternary ammonium compounds; phosphonium surfactants; sulfonium surfactants.

Examples of suitable zwitterionic surfactants may include N-alkyl derivatives of amino acids (such as glycine, betaine, aminopropionic acid); imidazoline surfactants; amine oxides; amidobetaines.

In one example, the surfactant may be a mixture of sodium dodecyl sulfate (CH₃(CH₂)₁₁OSO₃Na, also known as sodium lauryl sulfate) and polyvinyl alcohol ([CH₂CH(OH)]ₙ). In an example where the surfactant is a mixture of sodium dodecyl sulphate and polyvinyl alcohol, the mass ratio of sodium dodecyl sulphate to polyvinyl alcohol may be, for example, between 20:80 and 80:20, alternatively between 30:70 and 70:30, alternatively between 35:65 and 60:40. In a particular example, the mass ratio of sodium dodecyl sulphate to polyvinyl alcohol is 40:60.

The nano-diamond particles may be produced by any suitable means. For example, nano-diamond particles used in various examples may be produced by detonation methods, by crushing diamond grit particles synthesised by high-pressure/high-temperature (“HPHT”) processes, or by any other alternative method.

In one example, the size of the nano-diamond particles may be in the size range 1 nm to 500 nm. In some examples, the size of the nano-diamond particles may be in the size range 2 nm to 100 nm and in other examples the size of the nano-diamond particles may be in the size range 5 nm to 80 nm.

In some examples, the surfaces of the nano-diamond particles may be treated such that the surfaces are terminated with a chemical species. Species may be, for example, amine (—NR₂), hydrogen (—H), or carboxyl (—RCOOH). Further examples of species suitable for termination the nano-diamond particles may include alkane (—RH), alkene, (—RR′C═CRR′), alkyene (—RC≡CR), arene (—Ar), alkyl halide (—RX), aryl halide (—ArX), alcohol (—ROH), phenol (—ArOH), ether (—ROR′), aldehyde (—RCHO), ketone (—RR′C═O), ester (—RCOOR′), amide (—RC═ONHR′), nitrile (—RC≡N), and nitro (—ArNO₂). Surface termination may be used to alter the surface chemistry of the nano-diamond particles and consequently the behaviour of the particles in colloids and suspensions.

The surface termination may affect the zeta potential of the nano-diamond particles relative to the medium in which the nano-diamond particles are dispersed. Zeta potential is an indicator of the stability of a colloidal dispersion (that is the resistance of the colloidal dispersion to aggregating). The more the zeta potential deviates from zero (either positively or negatively), the more stable the colloidal dispersion. Typically a colloidal dispersion with a zeta potential of ±40 mV to ±60 mV (that is, between +40 mV and +60 mV or −40 mV and −60 mV) has good stability.

In some examples, the zeta potential of the nano-diamond particles when dispersed in the surfactant-deionised water solution may deviate from zero by between ±30 and ±60 mV (that is between +30 mV and +60 mV or between −30 mV and −60 mV). Alternatively the zeta potential of the nano-diamond particles when dispersed in the surfactant-deionised water solution may deviate from zero by between ±40 and ±60 mV (that is between +40 and +60 mV or between −40 mV and −60 mV).

Examples of active pharmaceutical ingredients that may be formulated into compositions for delivery by the methods disclosed herein may include (but are not limited to) ketoprofen, ibuprofen, Triclosan™, artermisinin, intraconazole and ciprofloxacin.

According to one example, a method of particle sizing for the dispersed products employs a Dynamic Light Scattering (“DLS”) instrument (Nano S, manufactured by Malvern Instruments UK). Specifically, the Malvern Instruments Nano S uses a red (633 nm) 4 mW Helium-Neon laser to illuminate a standard optical quality UV cuvette containing a suspension of the particles to be sized. The particle sizes quoted in this disclosure are those obtained with that apparatus using the standard protocol provided by the instrument manufacturer. The size of the nano-particles in a dry solid material are determined from the measurement of the particle size subsequent to the dry solid material being dispersed in water.

The effectiveness of the active pharmaceutical ingredient may be measured by means of its “half maximal inhibitory concentration”, also known as the “IC₅₀” value.

The IC₅₀ of a substance is a measure of the effectiveness of that substance in inhibiting a specific biological or biochemical function. The IC₅₀ indicates how much of a particular drug or other substance (inhibitor) is needed to inhibit a given biological process (or component of a process, i.e. an enzyme, cell, cell receptor or microorganism) by half. It is commonly used as a measure of antagonist drug potency in pharmacological research. The IC₅₀ of a drug can be determined by constructing a dose-response curve and examining the effect of different concentrations of antagonist on reversing agonist activity. IC₅₀ values can be calculated for a given antagonist by determining the concentration needed to inhibit half of the maximum biological response of the agonist.

Various versions may be formed according to one or more of the following examples which are presented as examples only and are not intended to be limiting.

### Example 1

0.1 g ketoprofen (Sigma Aldrich, ≥98% purity) [(RS)2-(3-benzoylphenyl)-propionic acid (chemical formula C₁₆H₁₄O₃)], a non-steroidal anti-inflammatory drug] was dissolved into 2 ml dimethyl sulfoxide (DMSO, Sigma Aldrich, analytical grade). 0.1 g cetyl trimethyl ammonium bromide (“CTAB”, from Sigma Aldrich) was dissolved into 5 ml deionized water using gentle heat to accelerate the dissolution of the CTAB. 0.05 g of nano-diamond particles (obtained from Carbodeon Ltd Oy, Finland) with an average particle size of approximately 8 nm as measured by the supplier and having surfaces modified such that the surfaces were terminated by amine groups (—NH₂ groups), were added to the CTAB-water solution and ultrasonically dispersed to form a homogeneous nano-diamond dispersion. After the nano-diamond particles were dispersed in the CTAB-water solution, the zeta potential was measured at room temperature (using a Malvern Zetasizer Nano S) to be +52 mV, indicating that the dispersion had good stability. The ketoprofen-DMSO solution was then added drop-wise into the nano-diamond particle dispersion whilst mechanically stirring the solution at a rate of approximately 300 rpm. Once all the ketoprofen-DMSO solution had been added to the nano-diamond particle dispersion, the stirring rate was reduced to approximately 60 rpm and stirring continued for an additional 30 minutes to form a final dispersion. The final dispersion was then subjected to a freeze drying process to obtain a ketoprofen/nano-diamond dry powder. The freeze-dried ketoprofen/nano-diamond powder was then dispersed back into deionized water. Measurement of the average size of the ketoprofen/nano-diamond particles was performed using a particle size measuring system (Malvern Instruments, Nano S); the average size was found to be approximately 189 nm.

### Comparative Example 1

0.1 g ketoprofen was dissolved into 2 ml dimethyl sulfoxide (DMSO). 0.1 g cetyl trimethyl ammonium bromide (CTAB) was dissolved into 5 ml deionized water using gentle heat to accelerate the dissolution of the CTAB. The ketoprofen-DMSO solution was then added drop-wise into the CTAB-water solution whilst mechanically stirring the solution at a rate of approximately 300 rpm. Once all the ketoprofen-DMSO solution had been added to the CTAB-water solution, the stirring rate was reduced to approximately 60 rpm and stirring continued for an additional 30 minutes to form a final dispersion. The final dispersion was subject to a freeze drying process to obtain a ketoprofen dry powder. The freeze-dried ketoprofen powder was then dispersed back into deionized water and insoluble particle were observed. Measurement of the average size of the ketoprofen particles was performed using a particle size measuring system (Malvern Instruments, Nano S); the average size was found to be greater than 2100 nm.

### Example 2

0.1 g ketoprofen [(RS)2-(3-benzoylphenyl)-propionic acid (chemical formula C₁₆H₁₄O₃)), a non-steroidal anti-inflammatory drug] was dissolved into 2 ml dimethyl sulfoxide (DMSO). 0.1 g cetyl trimethyl ammonium bromide (CTAB) was dissolved into 5 ml deionized water using gentle heat to accelerate the dissolution of the CTAB. 0.05 g of nano-diamond particles (obtained by crushing diamond synthesised by an HPHT process, supplied by Engis UK Limited, Henley-on-Thames) with an average particle size of approximately 50 nm and having surfaces of the nano-diamond particles that were modified such that the surfaces were terminated by hydrogen (—H termination), were added to the CTAB-water solution and ultrasonically dispersed to form a homogeneous nano-diamond dispersion. After the nano-diamond particles were dispersed in the CTAB-water solution, the zeta potential was measured to be +46 mV, indicating that the dispersion had good stability. The ketoprofen-DMSO solution was then added drop-wise into the nano-diamond particle dispersion whilst mechanically stirring the solution at a rate of approximately 300 rpm. Once all the ketoprofen-DMSO solution had been added to the nano-diamond particle dispersion, the stirring rate was reduced to approximately 60 rpm and stirring continued for an additional 30 minutes to form a final dispersion. The final dispersion was subjected to a freeze drying process to obtain a ketoprofen/nano-diamond dry powder. The freeze-dried ketoprofen/nano-diamond powder was then dispersed back to deionized water. Measurement of the average size of the ketoprofen/nano-diamond particles was performed using a particle size measuring system (Malvern Instruments, Nano S); the average size was found to be approximately 167 nm.

### Example 3

0.1 g ibuprofen (Sigma Aldrich, ≥98% purity) [(RS)-2-(4-(2-methylpropyl)phenyl) propanoic acid, a non-steroidal anti-inflammatory drug] was dissolved into 3 ml dimethyl sulfoxide (DMSO). 0.1 g cetyl trimethyl ammonium bromide (CTAB) was dissolved into 5 ml deionized water using gentle heat to accelerate the dissolution of the CTAB. 0.05 g of nano-diamond particles (obtained by crushing diamond synthesised by an HPHT process) with an average particle size of approximately 50 nm (same source as Example 2) and having surfaces modified such that the surfaces were terminated by hydrogen (—H termination), were added to the CTAB-water solution and ultrasonically dispersed to form a homogeneous nano-diamond dispersion. After the nano-diamond particles were dispersed in the CTAB-water solution, the zeta potential was measured to be +46 mV, indicating that the dispersion had good stability. The ibuprofen-DMSO solution was then added drop-wise into the nano-diamond particle dispersion whilst mechanically stirring the solution at a rate of approximately 300 rpm. Once all the ibuprofen-DMSO solution had been added to the nano-diamond particle dispersion, the stirring rate reduced to approximately 60 rpm for an additional 30 minutes. The final dispersion was subjected to a freeze drying process to obtain a ibuprofen/nano-diamond dry powder. The freeze-dried ibuprofen/nano-diamond powder was then dispersed back into deionized water. Measurement of the average size of the ibuprofen/nano-diamond particles was performed using a particle size measuring system (Malvern Instruments, Nano S); the average size was found to be approximately 99 nm.

### Comparative Example 3

0.1 g ibuprofen was dissolved into 3 ml dimethyl sulfoxide (DMSO). 0.1 g cetyl trimethyl ammonium bromide (CTAB) was dissolved into 5 ml deionized water using gentle heat to accelerate the dissolution of the CTAB. The ibuprofen-DMSO solution was then added drop-wise into the CTAB-water solution whilst mechanically stirring the solution at a rate of approximately 300 rpm. Once all the ibuprofen-DMSO solution had been added to the CTAB-water solution, the stirring rate was reduced to approximately 60 rpm and stirring continued for an additional 30 minutes. The final dispersion was subjected to a freeze drying process to obtain an ibuprofen dry powder. The freeze-dried ketoprofen powder was then dispersed back into deionized water and insoluble particles were observed. Measurement of the average size of the ibuprofen particles was performed using a particle size measuring system (Malvern Instruments, Nano S); the average size was found to be greater than 2800 nm.

### Example 4

0.1 g ibuprofen (Sigma Aldrich, ≥98% purity) [(RS)-2-(4-(2-methylpropyl)phenyl) propanoic acid), a non-steroidal anti-inflammatory drug] was dissolved into 3 ml dimethyl sulfoxide (DMSO). 0.16 g cetyl trimethyl ammonium bromide (CTAB) was dissolved into 5 ml deionized water using gentle heat to accelerate the dissolution of the CTAB. 0.05 g of nano-diamond particles (obtained by crushing diamond synthesised by an HPHT process) with an average particle size of approximately 50 nm from the same source as in Example 2 and having surfaces modified such that the surfaces were terminated by hydrogen (—H termination), were added to the CTAB-water solution and ultrasonically dispersed to form a homogeneous nano-diamond dispersion. After the nano-diamond particles were dispersed in the CTAB-water solution, the zeta potential was measured to be +46 mV, indicating that the dispersion had good stability. The ketoprofen-DMSO solution was then added drop-wise into the nano-diamond particle dispersion whilst mechanical stirring the solution at a rate of approximately 300 rpm. Once all the ketoprofen-DMSO solution had been added to the nano-diamond particle dispersion, the stirring rate was reduced to approximately 60 rpm and stirring continued for an additional 30 minutes. The final dispersion was subjected to a freeze drying process to obtain a ketoprofen/nano-diamond dry powder. The freeze-dried ketoprofen/nano-diamond powder was then dispersed back into deionized water. Measurement of the average size of the ketoprofen/nano-diamond particles was performed using a particle size measuring system (Malvern Instruments, Nano S); the average size was found to be approximately 94 nm.

### Example 5

0.1 g Triclosan™ (supplied by Sigma Aldrich) [5-chloro-2-(2,4-dichloro-phenoxy)-phenol)] was dissolved into 3 ml ethanol. 0.08 g sodium dodecyl sulphate (“SDS”, supplied by Sigma Aldrich, ≥99.0% purity) and 0.03 g polyvinyl alcohol (“PVA”, supplied by Sigma Aldrich, molecular weight approximately 9000, 80% hydrolysed,) was dissolved into 5 ml deionized water using gentle heat to accelerate the dissolution of the CTAB. 0.05 g of nano-diamond particles (obtained from Carbodeon Ltd Oy, Finland) with an average particle size of approximately 6 nm and having surfaces modified such that the surfaces were terminated by carboxyl groups (—COOH termination), were added to the SDS-PVA-water solution and ultrasonically dispersed to form a homogeneous nano-diamond dispersion.

After the nano-diamond particles were dispersed in the SDS-PVA-water solution, the zeta potential was measured to be −60 mV, indicating that the dispersion had good stability. The Triclosan™-ethanol solution was then added drop-wise into the nano-diamond particle dispersion whilst mechanically stirring the solution at a rate of approximately 300 rpm. Once all the Triclosan™-ethanol solution had been added to the nano-diamond particle dispersion, the stirring rate was reduced to approximately 60 rpm and stirring continued for an additional 30 minutes to form a final dispersion. The final dispersion was subject to a freeze drying process to obtain a Triclosan™/nano-diamond dry powder. The freeze-dried Triclosan™/nano-diamond powder was then dispersed back into deionized water. Measurement of the average size of the Triclosan™/nano-diamond particles was performed using a particle size measuring system (Malvern Instruments, Nano S); the average size was found to be approximately 94 nm.

### Example 6

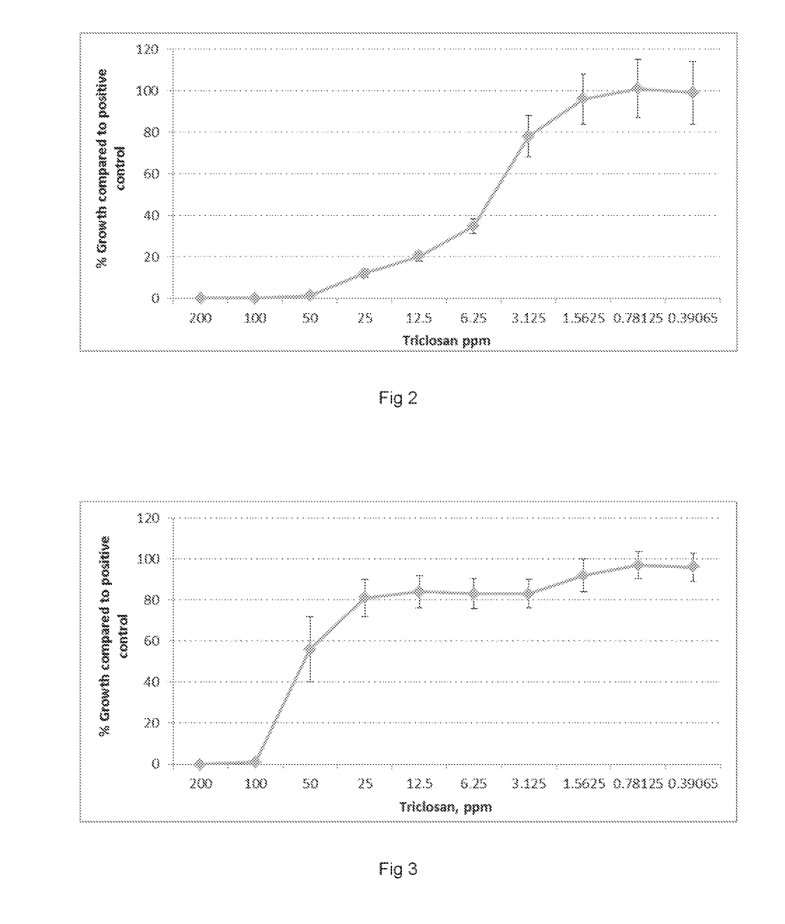

A sample of the Triclosan™/nano-diamond powder produced according to Example 5 was dispersed into deionized water to form an 800 ppm Triclosan™ dispersion (Solution 1). The half maximal inhibitory concentration (often referred to as an “IC₅₀ test” was determined for Solution 1 on a gram-negative bacterium (Fusobacterium nucleatum). The results of the IC₅₀ test are shown in FIG. 2, where the IC₅₀ was determined to be around 4.5 ppm. The results in FIG. 2 show percentage growth compared to positive control against Triclosan™ ppm.

### Comparative Example 6

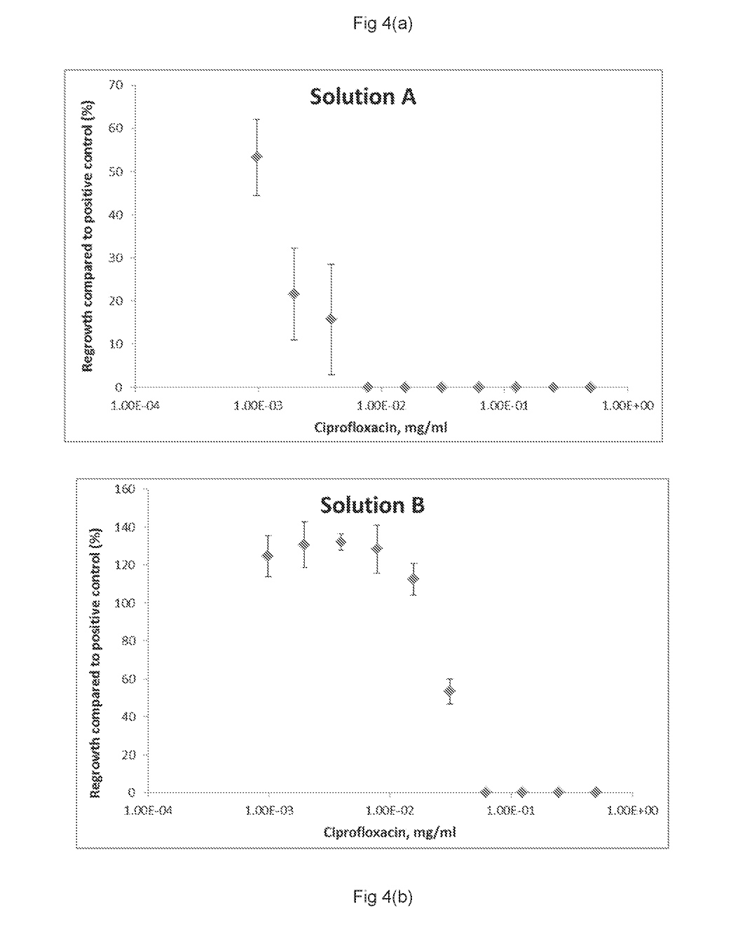

Triclosan™ was dissolved into an ethanol/water mixture (50/50 in volume) to form an 800 ppm Triclosan™ solution (Solution 2). An IC₅₀ test was conducted with Solution 2 on gram-negative bacteria (Fusobacterium nucleatum). The results of the IC₅₀ test are shown in FIG. 3, where the IC₅₀ was determined to be around 56 ppm. The results in FIG. 3 show percentage growth compared to positive control against Triclosan™ ppm.

### Example 7

70 mg of ciprofloxacin (purchased from Sigma Aldrich) was stirred in to 35 ml of chloroform, CHCl₃, (purchased from Sigma Aldrich, ≥99% purity) for approximately 1 hour, together with additional an 14 ml of 1-propanol (purchased from Sigma Aldrich, ≥99.5% purity). The mixture was stirred until all the ciprofloxacin was dissolved. 20 mg of polyvinyl alcohol (purchased from Sigma Aldrich, molecular weight approximately 9000, 80% hydrolysed) and 5 mg of sodium dodecyl sulphate (purchased from Sigma Aldrich, ≥99.0% purity) were dissolved in 50 ml of water together with 5 mg nano-diamond particles (particle size approximately 5 nm, obtained from Carbodeon Ltd Oy with surface terminated by carboxyl groups). The aqueous solution (polymer/surfactant) was transferred to the organic solution (active) and the resulting mixture was sonicated for approximately 1 minute using an ultrasonic processor. The emulsion was then spray-dried (Buchi Mini-290) at 150° C. inlet temperature and 10% pump rate. The resulting powder was dispersed into deionised water at concentration of 0.5 mg/ml. The average particle size was measured after dispersion with a Malvern Nano-S particle sizing machine and the found to be 188 nm.

### Comparative Example 7

70 mg ciprofloxacin (the active pharmaceutical ingredient) was stirred into 35 ml chloroform for approximately 1 hour together with an additional 14 ml of 1-propanol. The mixture was stirred until all the ciprofloxacin was dissolved.

24 mg of polyvinyl alcohol (purchased from Sigma Aldrich, molecular weight approximately 9000, 80% hydrolysed) and 6 mg of sodium dodecyl sulphate (purchased from Sigma Aldrich, ≥99.0% purity) were dissolved in 50 ml of water to form an aqueous solution.

The aqueous solution (polymer/surfactant) was transferred to the organic solution containing the active pharmaceutical ingredient and the resulting mixture was sonicated for 55 seconds with power 5 using the ultrasonic processor XL. The emulsion was then spray-dried (Buchi Mini-290) at 150° C. inlet temperature and 10% pump rate. The resulting powder was found to be non-dispersible in deionised water.

### Example 8

In this example, the efficacy of the product of Example 7 was tested against Comparative Example 7 using a Minimum Bactericidal Concentration Test (MBC) to compare the inhibitory.

A sample of the spray dried powder obtained from Example 7 was dispersed into deionised water to give a ciprofloxacin concentration of 0.5 mg/ml (“Solution A”). A dispersion having the same concentration of ciprofloxacin was prepared by dissolving ciprofloxacin into dimethyl sulfoxide (“Solution B”). A third sample (“Solution C”) consisting of a water saturated ciprofloxacin solution was prepared by dispersing powder obtained from Comparative Example 7.

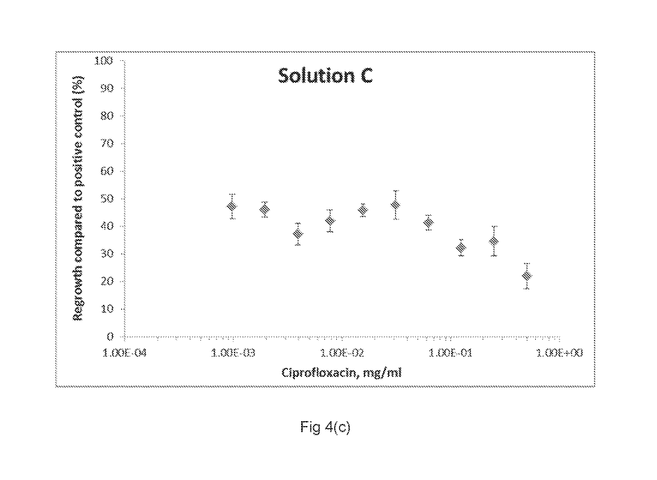

All three solutions were diluted to various concentrations and were subject to Minimum Bactericidal Concentration Test (MBC) to compare the inhibitory activity via re-growth of Staphylococcus aureus subsp. aureus MRSA252 as percentage of positive control. The results are shown in FIGS. 4a, 4b and 4c. The results showed that Solution A (nanodiamond aided ciprofloxacin nanoparticle dispersion) maintained activity against MRSA252 at 7.81 mg/L (see FIG. 4(a)). Solution B (DMSO/ciprofloxacin organic solution) maintained activity against MRSA252 at 62.5 mg/L (see FIG. 4(b)). Solution C (saturated solution of Comparative Example 7) did not show any activity against MRSA252 (see FIG. 4(c)).

While various versions have been described with reference to a number of examples, those skilled in the art will understand that various changes may be made and equivalents may be substituted for elements thereof and that these examples are not intended to limit the particular examples or versions disclosed.

What is claimed is:

1. A method for the preparation of a delivery drug delivery system comprising one or more active pharmaceutical ingredients having solubility in water of less than 1 g in 30 ml of water and nano-diamond, the method comprising the steps of:
(a) dissolving the active pharmaceutical ingredient(s) into a polar non-aqueous solvent to form a first mixture;
(b) dissolving a surfactant in deionized water to form a surfactant solution;
(c) adding a plurality of nano-diamond particles to the surfactant solution to disperse the nano-diamond particles in the surfactant solution thereby forming a nano-diamond dispersion;
(d) adding the first mixture to the nano-diamond dispersion whilst agitating the dispersion to form a second mixture; and
(e) drying the second mixture to produce a dry powder for use as a drug delivery system.

2. A method according to claim 1 wherein the step of dissolving the active pharmaceutical ingredient into a polar non-aqueous solvent to form a first mixture comprises dissolving the active pharmaceutical ingredient in a solvent comprising dimethyl sulfoxide.

3. A method according to claim 1 wherein the step of dissolving the active pharmaceutical ingredient into a polar non-aqueous solvent to form a first mixture comprises dissolving the active pharmaceutical ingredient in a solvent comprising ethanol.

4. A method according to claim 1, wherein the concentration of the active pharmaceutical ingredient in the polar non-aqueous solvent is in the range 1 g/dm³ to 200 g/dm³.

5. A method according to claim 1 wherein the step of dissolving a surfactant in deionized water to form a surfactant solution comprises dissolving cetyl trimethyl ammonium bromide ((C₁₆H₃₃)N(CH₃)₃Br) in deionized water.

6. A method according to claim 1 wherein the step of dissolving a surfactant in deionized water to form a surfactant solution comprises dissolving sodium dodecyl sulfate and polyvinyl alcohol in deionized water.

7. A method according to claim 1 where the concentration of the surfactant in deionized water is in the range 1 g/dm³ to 100 g/dm³.

8. A method according to claim 1 wherein the concentration of nano-diamond particles dispersed in the surfactant dissolved in deionized water is in the range 1 g/dm³ to 100 g/dm³.

9. A method according to claim 1 wherein the step of adding a plurality of nano-diamond particles to the surfactant solution to disperse the nano-diamond particles in the surfactant solution comprises adding a plurality of nano particles having an average grain size of between 1 nm to 200 nm.

10. A method according to claim 1, wherein the step of adding the first mixture to the nano-diamond dispersion comprises adding the first mixture drop-wise to the nano-diamond dispersion whilst mechanically agitating the dispersion to form the second mixture.

11. A method according to claim 1 wherein the step of drying the second mixture to produce a dry powder for use as a drug delivery system comprises freeze-drying.

12. A composition for medical treatment comprising:
a plurality of nano-diamond particles; and
an active pharmaceutical ingredient having a solubility in water of less than 1 g in 30 ml of water wherein
the composition is a dry powder having an average particle size in the range 500 nm or less when dispersed in deionised water.

13. A composition for medical treatment according to claim 12 wherein the active pharmaceutical ingredient comprises any one or more of ketoprofen, ibuprofen, Triclosan™, artermisinin, intraconazole and ciprofloxacin.

14.-15. (canceled)
